(19) United States
(12) Patent Application Publication
(10) Pub. No.: US 2009/0298106 A1
(43) Pub. Date: Dec. 3, 2009

(54) Methods for Monitoring Immunosuppressant Drug Levels, Renal Function, and Hepatic Function Using Small Volume Samples

(75) Inventor: Pleasant Fite Hooper, New Orleans, LA (US)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: Therapeutic Monitoring Services, L.L.C., New Orleans, LA (US)

(21) Appl. No.: 12/476,013

(22) Filed: Jun. 1, 2009

Related U.S. Application Data
(60) Provisional application No. 61/057,811, filed on May 30, 2008.

(51) Int. Cl. C12Q 1/52; C12Q 1/37; C12Q 1/32; C12Q 1/34; B01L 3/00; G01N 33/72; G01N 33/50

(52) U.S. Cl. 435/16; 435/24; 435/26; 435/18; 422/61; 436/97; 436/96; 436/161

(57) ABSTRACT

Systems and methods are provided for monitoring a immunosuppressant drug level and renal function, hepatic function, or a combination thereof in a patient, comprising obtaining a small volume blood sample from the patient; determining the level of at least one immunosuppressant drug in the small volume blood sample and determining the level of a second immunosuppressant drug or analyzing the renal function, the hepatic function, or a combination thereof in the patient. In some embodiments, the immunosuppressant drug levels are determined using a liquid chromatography tandem mass spectrometry (LC-MS/MS) procedure. Also provided are kits for use in any of the systems and methods described herein.

### PRIOR RELATED APPLICATION

The present application claims the benefit of priority to U.S. Provisional Application No. 61/057,811 filed May 30, 2008, which is hereby incorporated by reference in its entirety.

### FIELD OF THE INVENTION

The present disclosure relates to methods, kits, and systems for the efficient monitoring of the levels of immunosuppressant drugs, renal function, and/or hepatic function in a patient. In particular, the immunosuppressant drug levels, renal function, and/or hepatic function are monitored by using a small volume sample.

### BACKGROUND OF THE INVENTION

Humans have a complex immune response system to distinguish between native and foreign material, and the proper functioning of the immune system is vital for the long term health of the body. A deficient immune response can lead to a body's inability to protect itself from foreign matter, and an excessive immune response can lead to the body's overreaction to what would otherwise be innocuous matter.

In certain circumstances, the immune system must be controlled in order to either augment a deficient response or suppress an excessive response. For example, when organs such as kidney, heart, lung, bone marrow, and liver are transplanted in humans, the body will sometimes reject the transplanted tissue by a process referred to as allograft rejection. In treating allograft rejection, the immune system is frequently suppressed in a controlled manner through drug therapy with immunosuppressant drugs including, but not limited to, cyclosporin, tacrolimus, sirolimus, mycophenolic acid, and everolimus. Immunosuppressant drugs are carefully administered to transplant recipients in order to help prevent allograft rejection of the foreign (i.e. non-self) tissue. Many of the immunosuppressant drugs require the measurement of concentrations with subsequent dosage adjustment to maximize efficacy while minimizing toxicity.

Post-transplant patient adherence to the immunosuppressant treatment greatly improves the likelihood of positive, long-term outcomes. Because organ transplantation is a physically, emotionally, and financially draining process, it is critical that everything is done to ensure that the patients are compliant with their treatment regimen. Patient noncompliance is a major cause of poor transplant outcome—failure to adhere to medication regimens can lead to acute rejection episodes, graft loss, and even death. Accordingly, regular monitoring of immunosuppressant drug blood levels is an essential component of the post transplant medical regimen. More intense monitoring can promote improved compliance and better outcomes. Monitoring, however, may involve significant travel time to a hospital or clinic and time absent from school or employment. Depending on the age of the patient, these inconveniences may be shared by the patient's parents or guardians.

Traditionally, immunosuppressant drug monitoring has involved immunoassays such as enzyme-linked immunoassays (ELISA) and microparticle enzyme immunoassays. However, these assays are deficient in that they do not always provide an accurate measure of the drug levels due to cross-reactivity of the antibodies with drug metabolites (See, e.g., Soldin et al., 2003, Arch. Pathol. Lab. Med. 127:19-22). Some researchers, therefore, have used a liquid chromatography-tandem mass spectrometry (LC-MS² or LC-MS/MS) procedure as a quantitative assay of certain immunosuppressant drugs (Taylor et al., 1996, Clinical Chemistry 42(2):279-85; Taylor et al., 1997, Clinical Chemistry 43:2189-90). The use of these methods demonstrates increased accuracy, however, it requires that the patient go to the hospital or clinic for the venipuncture collection of a blood sample. These methods have the additional disadvantage in that patients have indicated that blood collection by venipuncture is more painful than other types of blood collection, such as fingerprick or earlobe sampling.

Yonan et al. have described the use of a fingerprick sampling method for the collection of blood samples from transplant recipients for monitoring cyclosporin levels with liquid chromatography tandem mass spectrometry (LC-MS/MS)(Yonan et al., 2006, Clinical Transplantation 20:221-25). This method provides an accurate method for monitoring cyclosporin levels, with a wide analytical range (up to 5,000 μg/L) that precludes the need for the dilution of samples outside the analytical range as is seen in the standard immunoassay methods. The method also provides the opportunity for patients to collect their own blood sample at home, rather than being inconvenienced by having to travel to the hospital or clinic. However, this sampling method has only been used for the monitoring of a single immunosuppressant drug in the transplant patients. Because patients would typically be treated with several immunosuppressant drugs and because other tests are typically run from venipuncture samples in order to assess patient health, it would still be necessary for the patient to travel to the hospital or clinic to be assessed for those additional drug levels and/or standard tests.

What is needed, therefore, are methods for the regular, efficient, and accurate monitoring of immunosuppressant drug level(s) and renal function, hepatic function, or a combination thereof that are more convenient than the currently available methods and that facilitate patient compliance, improve patient prognosis, and improve quality of life. Also needed are kits for use in combination with the disclosed methods.

### SUMMARY OF THE INVENTION

The present disclosure solves these problems by providing methods for monitoring immunosuppressant drug levels, hepatic function, renal function, and hematologic biomarker levels in a patient using a small volume sample. In some embodiments, the methods may be used for monitoring the levels of at least two immunosuppressant drugs in a patient in need thereof. In other embodiments, the methods are used for monitoring the level of at least one immunosuppressant drug in a patient, as well as for analyzing the renal function or hepatic function in the patient. In yet other embodiments, the methods are used for monitoring the levels of at least one immunosuppressant drug in a patient, as well as for analyzing both renal function and hepatic function in the patient. In other embodiments, the methods are used for monitoring the levels of at least two immunosuppressant drugs in a patient, as well as for analyzing both renal function and hepatic function in the patient.

The methods comprise obtaining a small volume blood sample from a patient; determining the level of at least one immunosuppressant drug in the small volume blood sample; and determining the level of a second immunosuppressant drug, analyzing the renal function, analyzing the hepatic function, or a combination thereof in the small volume sample. In certain embodiments, the step of determining the immunosuppressant drug level is performed using a liquid chromatography tandem mass spectrometry (LC-MS/MS) procedure. In other embodiments, the step of determining the immunosuppressant drug level is performed using high performance liquid chromatography coupled to electro-spray ionization tandem mass spectrometry (HPLC-ESI-MS/MS). In one preferred embodiment, the methods are used to monitor the level of at least one immunosuppressant drug and the renal function in the patient.

In some embodiments, the present methods are used for monitoring immunosuppressant drug levels and indicators of patient health in a patient who has had or is going to have an organ transplant. The organ is selected from the group consisting of, but not limited to, liver, kidney, heart, lung, pancreas, and bone marrow. The immunosuppressant drugs, in certain embodiments, are selected from the group consisting of cyclosporin, tacrolimus, sirolimus, mycophenolic acid, and everolimus. In one embodiment, the levels of at least two of cyclosporin, tacrolimus, sirolimus, mycophenolic acid, and everolimus are analyzed from the same small volume blood sample. In one embodiment, the amount of the small volume sample used for a particular test or assay is 50 μL. In certain embodiments, the small volume blood sample is a fingerprick sample or an earlobe sample. The small volume blood sample is obtained, in some embodiments, by the patient. In other embodiments, the small volume sample is obtained by a healthcare professional.

In some embodiments, the patient's renal function, hepatic function, or both also are monitored using a small volume blood sample. The renal function is monitored, in some embodiments, by determining the glomerular filtration rate, by determining the creatinine clearance rate, or by determining the level of at least one of creatinine and urea. In one embodiment, the renal function is monitored by determining the level of creatinine, urea, or both in the small volume sample. The hepatic function is monitored, in some embodiments, by determining the levels of aspartate transaminase (AST), alanine transaminase (ALT), alkaline phosphatase (AP), total bilirubin (TBIL), direct bilirubin, gamma glutamyl transpeptidase (GGT), 5′ nucleotidase (5′NTD), serum glucose, lactate dehydrogenase, or a combination thereof. In one embodiment, the patient's hepatic function is monitored by determining the levels of each of the indicators AST, ALT, AP, TBIL, and direct bilirubin. In another embodiment, the hepatic function is monitored by determining the levels of AST, ALT, AP, TBIL, direct bilirubin, or a combination thereof. In certain embodiments, immunosuppressant drug levels are assayed by LC-MS/MS; hepatic function and/or renal function are assayed by standard chemical assays; and the results for the immunosuppressant drug levels, the renal function, and/or hepatic function are all reported to the patient or a health care provider in a single report.

Kits are also provided for use in any of the methods described herein. In certain embodiments, the kit comprises: an alcohol swab; a device for obtaining a small volume blood sample, such as a fingerprick device; a container for the collection of a small volume blood sample comprising an anti-clotting agent, a preservative agent, or both; a package for transport of the small volume blood sample; and instructions. The instructions may explain the particulars for collecting and packaging the small volume blood sample, and for transporting the small volume blood sample to a testing facility that analyzes the small volume blood sample for the level of at least one immunosuppressant drug and a second immunosuppressant drug, renal function, hepatic function, or a combination thereof. In certain embodiments, the device for obtaining a small volume blood sample is a lancet. In certain embodiments, the anti-clotting agent in the container for collection is dipotassium ethylenediaminetetraacetic acid (K₂-EDTA).

Systems are provided for monitoring at least one immunosuppressant drug level and a second immunosuppressant drug level, renal function, hepatic function, or a combination thereof in a patient. The systems include obtaining a small volume blood sample from the patient; placing the small volume blood sample in a container for transport to a testing facility that analyzes the small volume blood sample for the level of at least one immunosuppressant drug and renal function, hepatic function, or a combination thereof; and receiving results from the testing facility for the level of at least one immunosuppressant drug and renal function, hepatic function, or a combination thereof. In some embodiments, the patient obtains his or her own blood sample. In certain embodiments, the small volume blood sample is a fingerprick sample or an earlobe sample. The patient may receive the results directly from the testing facility or indirectly, from a health care provider that received the results from the testing facility. In certain embodiments, the small volume blood sample is transported to the testing facility by a postal service.

### BRIEF DESCRIPTION OF THE FIGURES

FIG. 1 is a schematic illustration of one embodiment of the present methods.

FIG. 2 includes bar graphs that show that the use of fingerprick samples in the present methods generates results that are comparable to those obtained using venipuncture samples. FIGS. 2A, 2B, and 2C reflect measurements of tacrolimus, rapamycin, and cyclosporin A, respectively using either a fingerprick blood sample (right bar in each pair) or a venipuncture blood sample (left bar in each pair). The measurements are shown as μg/L on the y axis, and the numbers shown on the x-axis reflect the number of the comparison made between a simultaneously taken fingerprick sample and venipuncture sample from a number of different patients. For each pair of bars shown, the fingerprick sample and venipuncture sample were taken simultaneously from the same patient.

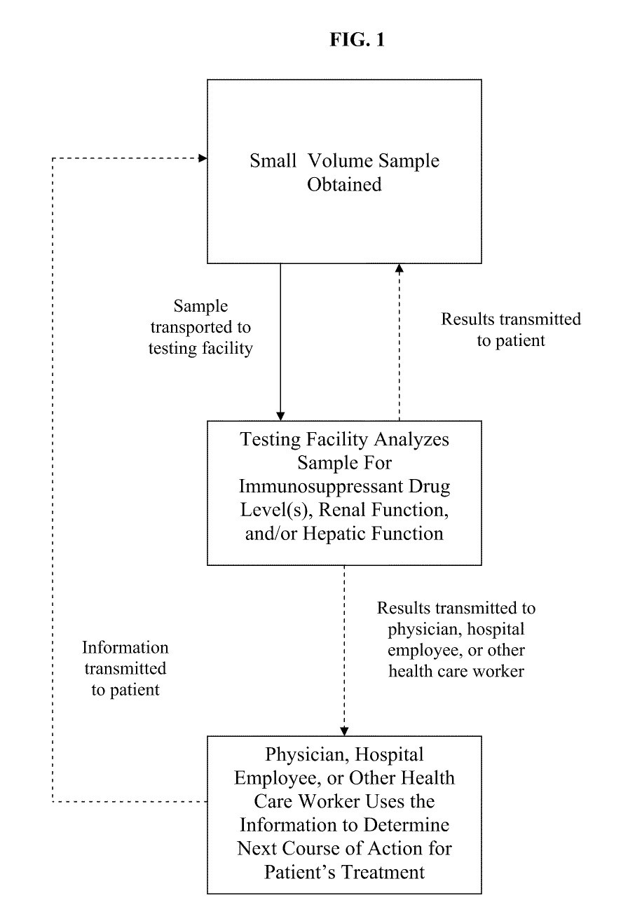

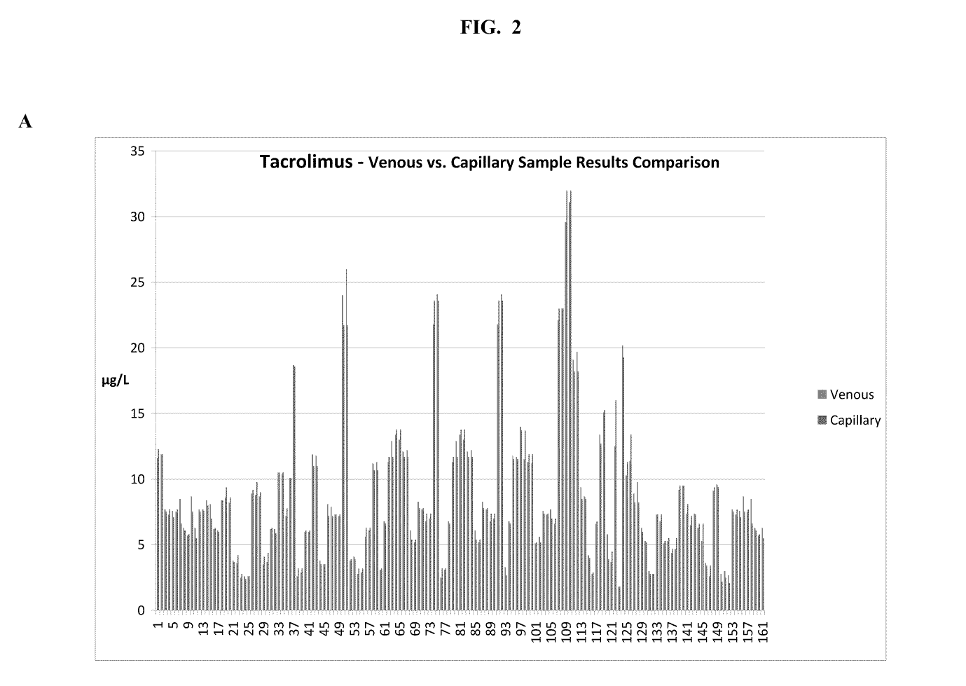

### DETAILED DESCRIPTION OF THE PRESENT INVENTION

The present invention may be understood more readily by reference to the following detailed description of the preferred embodiments of the invention and the Examples included herein. However, before the present methods are disclosed and described, it is to be understood that this invention is not limited to specific conditions, specific methods, etc., as such may, of course, vary, and the numerous modifications and variations therein will be apparent to those skilled in the art. It is also to be understood that the terminology used herein is for the purpose of describing specific embodiments only and is not intended to be limiting. It is further to be understood that unless specifically defined herein, the terminology used herein is to be given its traditional meaning as known in the relative art. As used in the specification and in the claims, “a” or “an” can mean one or more, depending upon the context in which it is used. Thus, for example, reference to “a sample” can mean that one or more than one sample can be utilized.

Methods are provided for monitoring immunosuppressant drug levels, hepatic function, renal function, and hematologic biomarker levels in a patient using a small volume sample. In some embodiments, the methods may be used for monitoring the levels of at least two immunosuppressant drugs in a patient in need thereof. In other embodiments, the methods are used for monitoring the level of at least one immunosuppressant drug in a patient, as well as for analyzing the renal function or hepatic function in the patient. In yet other embodiments, the methods are used for monitoring the levels of at least one immunosuppressant drug in a patient, as well as for analyzing both renal function and hepatic function in the patient. In yet other embodiments, the methods are used for monitoring the levels of at least two immunosuppressant drugs, as well as for analyzing both renal function and hepatic function in the patient.

The methods comprise obtaining a small volume blood sample from a patient; processing a portion of the blood sample, if necessary, for certain tests as described below; determining the level of at least one immunosuppressant drug in the small volume blood sample; and determining the level of a second immunosuppressant drug, analyzing the renal function, analyzing the hepatic function, or a combination thereof using the small volume sample or processed small volume sample. As used herein, the term “patient” refers to a human or an animal undergoing or having undergone some form of medical procedure, treatment, or testing. In some embodiments, the present methods are used for monitoring immunosuppressant drug levels in a patient who has had or is going to have an organ transplant. The organ may be selected from the group consisting of, but not limited to, liver, kidney, heart, lung, pancreas, and bone marrow. As also used herein, the term “immunosuppressant drug” is used to refer to an immunosuppressant drug or other compound or molecule used for the treatment of a patient who has had or is going to have an organ transplant. The term “therapeutic drug monitoring of immunosuppressant drugs” or “TDM-IS” is used to refer to the measurement of a therapeutic drug level of an immunosuppressant in the sample. TDM-IS is typically used for drugs that will lead to undertreatment or resistance if used at a deficient level, and can lead to toxicity and tissue damage if used at an excessive level. As used herein, the term “processed small volume sample” may refer to a plasma fraction (supernatant) of a small volume blood sample that is obtained by centrifuging a portion of the small volume blood sample.

The present methods have been described herein as being used for the analysis of a small volume whole blood sample. However, the use of various additional biological samples is contemplated by the inventors. For example, the methods may be useful for the analysis of other biological samples, such as plasma, serum, saliva, urine, biopsy, tissue, and other bodily fluid (e.g., amniotic fluid, spinal fluid, lymphatic fluid, mucus, and the like).

The immunosuppressant drug of the claimed methods, in certain embodiments, is selected from the group consisting of cyclosporin, tacrolimus, sirolimus, mycophenolic acid, and everolimus. In one embodiment, the levels of at least two immunosuppressant drugs are determined in the same small volume blood sample, wherein the immunosuppressant drugs are selected from the group consisting of cyclosporin, tacrolimus, sirolimus, mycophenolic acid, and everolimus. Cyclosporin and tacrolimus are calcineurin inhibitors. Cyclosporin (also referred to as cyclosporine or cyclosporin A) is a fungal peptide comprising eleven amino acids and is one of the most widely used immunosuppressive drugs. Cyclosporin is thought to bind to the cytosolic protein cyclophilin of immunocompetent lymphocytes, especially T-lymphocytes, producing a complex that inhibits calcineurin. Cyclosporin also inhibits lymphokine production and interleukin release, leading to a reduced function of effector T-cells. Cyclosporin is used in the treatment of acute rejection reactions, but has been increasingly substituted with newer immunosuppressants, as it is nephrotoxic.

Tacrolimus also is a fungal product and is a macrolide lactone which acts by inhibiting calcineurin similarly to cyclosporin. Tacrolimus is used particularly in liver and kidney transplantations, but also is used in heart and lung transplants. Tacrolimus is more potent than cyclosporin and has less-pronounced side effects. Sirolimus (also referred to as rapamycin) is a structurally related macrolide lactone, but functions differently and has different side effects. In contrast to the action by cyclosporine and tacrolimus, which affect the first phase of T lymphocyte activation, sirolimus affects the second phase of T lymphocyte activation, affecting signal transduction and clonal proliferation. Because sirolimus exerts its effect by a distinct mechanism, it acts synergistically with cyclosporine and, in combination with other immunosuppressants, has few side-effects. Sirolimus also indirectly inhibits T lymphocyte signal transduction and prevents B cell differentiation to the plasma cells. Everolimus is a derivative of Sirolimus that is currently approved for human use in Europe and Australia, and is in Phase III clinical trials in the United States.

Other immunosuppressant drugs that may be monitored in organ transplant patients using the disclosed methods include, but are not limited to, leflunomide, corticosteroids, azathioprine (IMURAN®), and monoclonal antibodies (OKT3®, ATGAM®, SIMULECT®, and ZENAPAX®), and more recently identified immunosuppressant drugs such as ISAtx247, Gusperimus (15-deoxyspergualin, SPANIDIN®), Medi-500 (formerly T10B9), FTY 720, Medi-507, and HLA-B2702 peptide (See, e.g., Gummert, 1999, J. Am. Soc. Nephrol. 10:1366-80; Gregory et al., 2004, Transplantation. 78(5):681-5; Nakanishi et al., 2007, Tohoku J. Exp. Med. 211:195-200; Brown et al., 1999, Drugs Res. Dev. 1(1):92-94; Aki and Kahan, 2003, Expert Opin. Biol. Ther. 3(4):665-81; Tarazona et al., 2000, J. Immunol. 165:6776-82).

In certain embodiments, the small volume blood sample is a fingerprick sample or an earlobe sample. The small volume blood sample is obtained, in some embodiments, by the patient. In other embodiments, the small volume sample is obtained by a healthcare professional. The sample is typically obtained by collecting the blood sample in a collection tube that contains an anti-clotting agent and optionally a preservative agent. The anti-clotting agent may include, for example, agents such as EDTA, lithium-heparin, and K₂-EDTA. The preservative agent may include glucose, phosphoenol pyruvate (PEP), mannitol, raffinose, hyaluronic acid (collagen), calpain inhibitor III, calpain inhibitor IV, glutaraldehyde, glutamine, and combinations thereof. In one embodiment, the preservative agent is 10 mM glutamine and 10 mM phosphate. Alternatively, the sample may be obtained by spotting the blood on a suitable filter paper as is known in the art. In certain embodiments, the sample is obtained by spotting the blood on a nitrocellulose membrane, filter paper, FTA paper (Whatman, Piscataway, N.J.), polytetrafluoroethylene (PTFE, TEFLON (DuPont, Wilmington, Del.), or polyvinylidene fluoride (PVDF).

As used herein, the phrase “small volume” is used to refer to a volume from about 10 μL to about 1,000 μL and depends on the number of tests to be performed on the sample. In certain embodiments, the sample is equal to or less than about 1,000 μL. In other embodiments, the sample is equal to or less than about 950, 900, 850, 800, 750, 700, 650, 600, 550, 500, 450, 400, 350, 300, 250, 200, 150, 100, or 50 μL. In one embodiment, the small volume sample is about 500 μL. By contrast, a typical venipuncture sample is 6 to 7 mL, approximately 12-14 times the amount used in the presently disclosed methods. The range of suitable sample sizes varies by the patient participating and the testing required for that patient. Generally, experienced patients, either from repeat sampling or history of diabetic fingerstick use, will provide about 500 μL on a routine basis in a microtube containing EDTA or other anti-clotting agent. A minimum of about 250 μL whole blood is needed to test both immunosuppressant drug levels and hepatic and/or renal function in the patient. A sample of that size may reduce the number of clinical chemistry tests that may be possible, in some cases. In other embodiments, a minimum of about 500, 450, 400, 350, 300, or 250 μL is required. In certain embodiments, the amount of sample typically used for simultaneous immunosuppressant drug monitoring is about 50 μL. In other embodiments, that sample is about 75, 70, 65, 60, 55, or 50 μL. In certain embodiments, the desired clinical chemistry tests (e.g., renal or hepatic function tests) are performed on the plasma fraction obtained after centrifugation of the sample volume left after about 50 μL has been taken for tandem mass spectrometry (MS/MS) tests and about 50 μL has been devoted to complete blood count (CBC) testing.

If immunosuppressant drug monitoring, complete blood count, renal function tests, and hepatic function tests are going to be conducted on a single sample volume collected in a container, then that single sample is separated into three fractions. In one embodiment, two samples of about 50 μL are removed—one for tandem mass spectrometry testing for immunosuppressant drug levels, and the other for complete blood count. The remaining volume is spun down in a nipple tube to obtain the plasma fraction, and that volume will vary depending on the particular blood sample characteristics. Preferably, approximately 200 μL of plasma is used to run both renal and hepatic tests for chemistry.

In certain embodiments, the collection tubes are about 1 mL in volume with a maximum collection line value of 500 μL. In addition, a variety of preservative agents may be included to extend the stability of a fresh blood sample during the time and temperature exposure of shipping. Such preservative agents may include, for example, glucose, phosphoenol pyruvate (PEP), mannitol, raffinose, hyaluronic acid (collagen), calpain inhibitor III, calpain inhibitor IV, glutaraldehyde, glutamine, and combinations thereof.

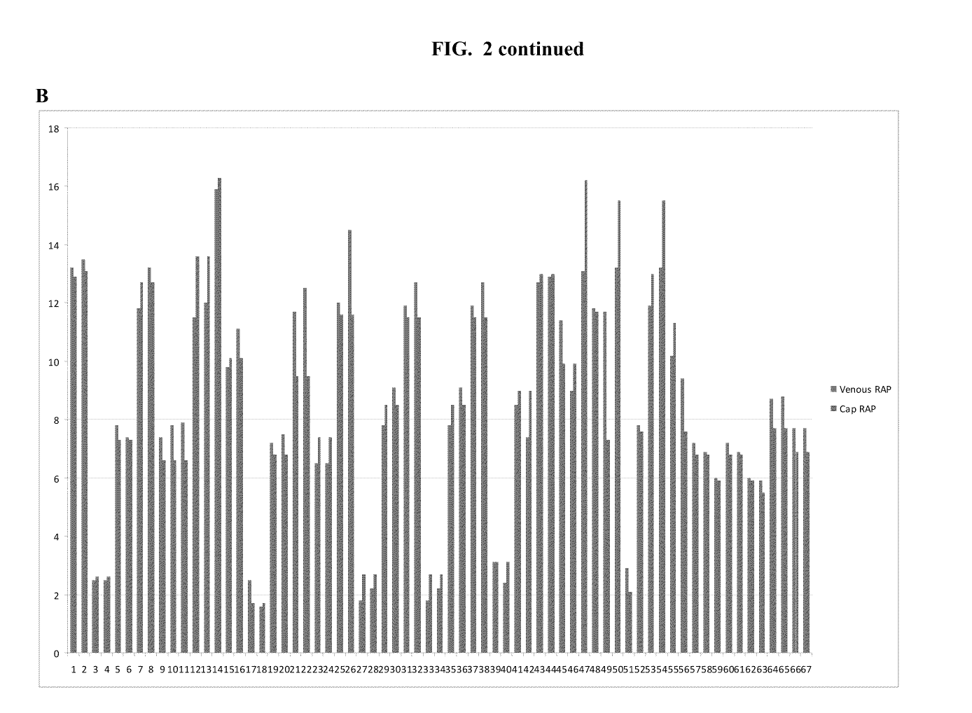

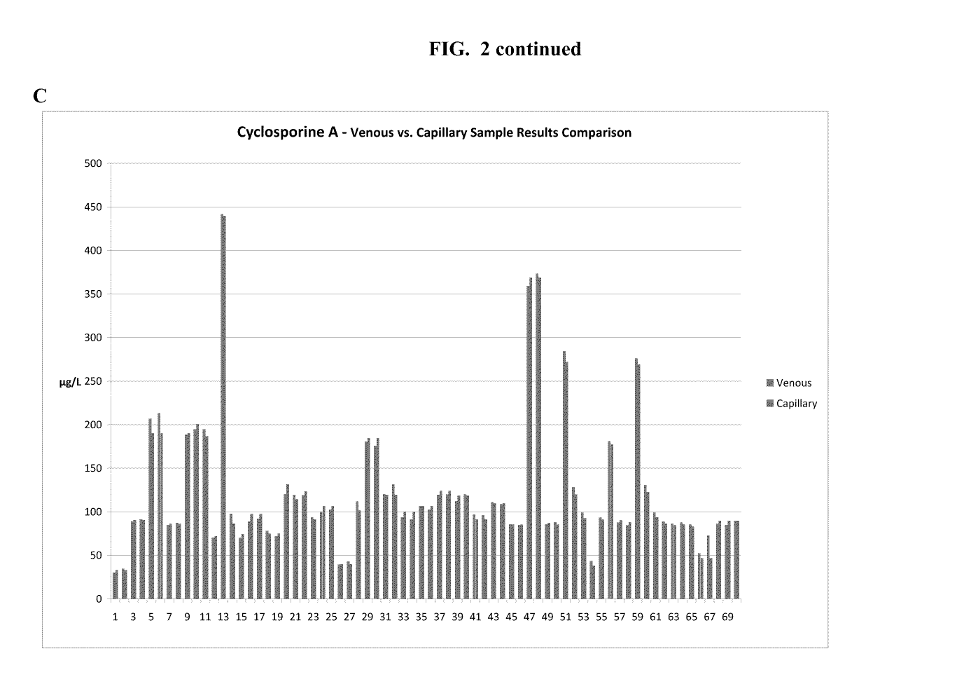

In certain embodiments, the step of determining an immunosuppressant drug level is performed using a liquid chromatography tandem mass spectrometry (LC-MS/MS) procedure. For a description of an LC-MS/MS procedure, see, for example, Taylor et al. (1996, Clinical Chemistry 42(2):279-85) and Taylor et al. (1997, Clinical Chemistry 43:2189-90). In particular, the determining step may be performed using high performance liquid chromatography coupled to electro-spray ionization tandem mass spectrometry (HPLC-ESI-MS/MS). The accuracy of this testing method using a fingerprick sample as compared to results obtained with a venipuncture sample was analyzed for tacrolimus, rapamycin, and cyclosporine A and was found to be comparable (FIG. 2). FIGS. 2A, 2B, and 2C show the results of analysis of the levels of tacrolimus, rapamycin, and cyclosporin A. The x-axis reflects the number of different comparisons that were made using samples obtained from several different patients. Each pair of bars shown represents the results obtained from a venipuncture sample (left bar) and a fingerprick sample (right bar) that were simultaneously taken from the same patient. It is contemplated that the samples may be assayed by various other mass spectrometry methods as well. For example, in some embodiments, matrix assisted laser desorption-ionization (MALDI) MS, surface enhanced laser desorption-ionization (SELDI), Time-of-Flight (TOF) MS, or some combination or variation thereof may be utilized. In addition, it is contemplated that the blood sample may be analyzed for other desired aspects, through the use of various polymerase chain reaction (PCR)-based assays.

In certain embodiments, the small volume blood sample is used to monitor at least one immunosuppressant drug level and is also used to monitor renal function, hepatic function, or a combination thereof. As used herein, the term “renal function” is used to describe the state of health of the patient's kidneys, including their excretory function, as determined by a test or assay described herein or as is well known in the art. The renal function may monitored, in some embodiments, by determining the glomerular filtration rate, by determining the creatinine clearance rate, or by determining the level of at least one waste substance of creatinine and urea. As used herein, the term “glomerular filtration rate” refers to the rate at which the kidneys filter the blood, removing excess wastes and fluids, and provides a calculation that is one way to measure remaining kidney function. GFR is calculated using a mathematical formula, as well known in the art, that compares a person's size, age, sex, and race to serum creatinine levels. A GFR under 60 mL/min/1.73 m² may be an indicator of decreased kidney function. In one embodiment, the renal function is monitored by determining the level of creatinine, blood urea nitrogen (BUN), or both in the sample using standard methods (See, e.g., Alfawassermann ACE Clinical Chemistry System Operator's Manual, August 2005 revision). If the kidney function is normal, the blood creatinine levels would fall within about 0.6 and about 1.2 mg/dL or about 53 to about 106 μmol/L for men, within about 0.5 and about 1.1 mg/dL or about 44 to about 97 μmol/L for women, within about 0.5 and about 1.0 mg/dL for teens, within about 0.3 and about 0.7 mg/dL for children, and within about 0.3 and about 1.2 mg/dL for newborns. If the kidney function is normal, the BUN to creatinine ratio is between about 10:1 and about 20:1 for patients over 12 months of age and up to about 30:1 for patients less than 12 months of age.

As used herein, the term “hepatic function” is used to refer to the state of health of the patient's liver, as determined by a test or assay described herein or as is well known in the art. The hepatic function is monitored, in some embodiments, by determining the levels of aspartate transaminase (AST), alanine transaminase (ALT), alkaline phosphatase (AP), total bilirubin (TBIL), direct bilirubin, gamma glutamyl transpeptidase (GGT), 5′ nucleotidase (5′NTD), serum glucose, lactate dehydrogenase, or a combination thereof using standard methods (See, e.g., Alfawassermann ACE Clinical Chemistry System Operator's Manual, August 2005 revision). In one embodiment, the patient's hepatic function is monitored by determining the levels of each of the indicators AST, ALT, AP, TBIL, and direct bilirubin. In another embodiment, the hepatic function is monitored by determining the levels of AST, ALT, AP, TBIL, direct bilirubin, or a combination thereof. If liver function is normal, the AST level should be within about 10 and about 40 IU/L; the ALT level should be within about 5 and about 40 IU/L; the AP level should be within about 30 and about 120 IU/L; the TBIL should be within about 2 to about 14 μmol/L; and the direct bilirubin level should be less than or about 4 μmol/L.

In one embodiment, the hepatic function and/or renal function is determined using a standard chemical analyzer as well known in the art. For example, the ACE System is a fully automated system, with an internal computer that directs all system functions, including test requisitioning, analyzer operation, data acquisition, data processing, report generation, and fault monitoring. (See Alfawassermann ACE Clinical Chemistry System Operator's Manual, August 2005 revision). Definitions for a wide variety of manufacturer-validated tests are included with the system and, for added flexibility, definitions for additional tests can be entered by the testing facilities. Reagents are maintained on the system at a temperature to maximize their on-system stability. Samples can be introduced into the system directly from primary tubes or from sample cups filled by the operator. An optional on-system barcode reader allows positive identification of bar-coded primary tubes, while sample cups are identified by means of a worklist prepared by the operator. The liquid level in each sample container is measured when the sample is aspirated, and the operator is notified when there is insufficient sample for analysis. Finally, the time each sample remains on the system is monitored and, for quality assurance purposes, a sample is not used after it has been on the system for more than two hours.

Kits are also provided for use in any of the methods described herein. In certain embodiments, the kit comprises: an alcohol swab, a device for obtaining a small volume blood sample such as a fingerprick device, a container for the collection of the small volume blood sample comprising a anti-clotting agent and optionally a preservative agent, a package for transport of the small volume blood sample, and instructions. The instructions may include instructions for the proper method to obtain the fingerprick or earlobe blood sample using the alcohol swab, fingerprick device, and collection vial, as well as instructions for the proper storage and shipping of the blood sample. In certain embodiments, suitable samples for the described methods have been stored at ambient temperature for less than about seventy-two, sixty, forty-eight, thirty-six, or twenty-four hours. In other embodiments, suitable samples have been stored at ambient temperature for about twenty-four hours or less, at about 4° C. for about seven days or less, or at about −20° C. for up to six months. Samples that are clotted, have inadequate volume, plasma, serum, or that have been at ambient temperature for greater than twenty-four hours without a preservative agent may, in some embodiments, not be suitable for the methods described herein. In certain embodiments, the device for obtaining the small volume blood sample is a lancet. The collection container typically will contain an anti-clotting agent and optionally a preservative. The anti-clotting agent may include, for example, EDTA, lithium-heparin, or K₂-EDTA. In certain embodiments, the anti-clotting agent in the container for collection is K₂-EDTA. The preservative agent may include, for example, glucose, phosphoenol pyruvate (PEP), mannitol, raffinose, hyaluronic acid (collagen), calpain inhibitor III, calpain inhibitor IV, glutaraldehyde, glutamine, or combinations thereof

Systems are provided for monitoring an immunosuppressant drug level and renal function, hepatic function, or a combination thereof in a patient. The systems include obtaining a small volume blood sample from the patient; placing the small volume blood sample in a container for transport to a testing facility that analyzes the small volume blood sample for the level of at least one immunosuppressant drug and renal function, hepatic function, or a combination thereof; and receiving results from the testing facility for the level of at least one immunosuppressant drug and renal function, hepatic function, or a combination thereof. In some embodiments, the patient obtains his or her own blood sample using a fingerprick device such as a lancet. In certain embodiments, the small volume blood sample is a fingerprick sample or an earlobe sample and is collected in a collection container containing an anti-clotting agent and optionally a preservative. In other embodiments, the sample is collected as a blood spot on a suitable filter paper.

The patient or healthcare provider that obtained the sample from the patient packages the collection tube or dried blood spot in a suitable package for shipment. In one embodiment, the package includes a foam insert in which the collection tube is directly inserted, a foam cap which is placed on top of the foam insert, an inner aluminum canister with screw cap, and an outer canister with screw cap. In certain embodiments, the small volume blood sample is transported to the testing facility by the national postal service. However, the sample also may be transported by other delivery services are suitable as well, including overnight shipment, second day delivery, and same day courier.

The testing facility or analytical lab receives the sample from the patient or healthcare provider and may perform initial processing of the sample as discussed above (e.g., separating the sample into several fractions and centrifuging certain fractions). The testing facility performs the simultaneous analysis of immunosuppressant drug levels and the analysis of various liver and/or hepatic markers of patient health. The various assays may be conducted a single time, in duplicate, or in triplicate. For monitoring of Tacrolimus, the Limit of Quantitation (LOQ) (i.e., functional sensitivity) is about 0.53 ng/mL or μg/L, with an upper range of about 45 μμL. For monitoring of Sirolimus (rapamycin) with the disclosed methods, the LOQ is about 0.88 μg/L, with an upper range of about 65 μg/L. For monitoring of Cyclosporine A with the disclosed methods, the LOQ is about 3 μg/L, with an upper range of about 1000 μg/L. For monitoring of Creatinine, the sensitivity is about 0.2 mg/dL up to about 14 mg/dL. For the monitoring of BUN, the sensitivity is about 0 mg/dL up to about 120 mg/dL. For the monitoring of AST, the sensitivity is about 7 U/L up to about 450 U/L. For the monitoring of ALT, the sensitivity is about 10 U/L up to about 600 U/L. For the monitoring of ALP, the sensitivity is about 2 U/L up to about 1400 U/L. For the monitoring of Direct Bilirubin, the sensitivity is about 0.0 mg/dL up to about 14.0 mg/dL. For the monitoring of Total Bilirubin, the sensitivity is about 0.0 mg/dL to about 40.0 mg/dL. For the monitoring of Albumin, the sensitivity is about 0.1 g/dL up to about 7.0 g/dL.

In addition, the results may be confirmed using a cross-platform quality control system. With multiple tests being done on the patient blood samples, there is always a question of accuracy of the results. Here, the high accuracy of the tandem mass spectrometer (MS/MS) may be used to benchmark performance of the standard clinical chemistry instrument. For example, the creatinine blood level can be determined by MS/MS in order to confirm the results obtained by standard chemistry analyzer. When the two results correlate, this supports the reliability of the results.

The patient may receive the results directly from the testing facility or indirectly, from a health care provider that received the results from the testing facility. If the health care provider receives the results from the testing facility, he or she may make a recommendation to take no action if the tested immunosuppressant drug levels are in the desired range, or may make a recommendation to modify the prescription of the immunosuppressant drug if the levels are outside the desired range. In other embodiments, the health care provider may make a recommendation to take no action if the tested renal or hepatic markers are within the normal range, or may make a recommendation to modify the prescription based on the results being out of the desired or normal range. Further, the health care provider may inquire about patient compliance, based on results obtained for immunosuppressant drug levels, renal function, hepatic function, or a combination thereof.

The present systems and methods are described with respect to the monitoring of immunosuppressant drug levels, hepatic function, and renal function, using a small volume blood sample. The present systems and methods are also particularly suitable to monitor compliance of patients participating in clinical trials for new immunosuppressant drugs. The methods decrease costs of the trials because the patients would not have to be compensated for the time and travel expenses and would allow for a greater demographic of participants in a particular trial as they would not have to be located close to the testing facility.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLES

### Example 1

Preparation of Small Volume Blood Sample from Fingerstick

A small volume blood sample is collected using the HOMETRAK (TMS Biosciences, New Orleans, La.) blood collection kit. The patient's hands are washed and then the desired area is cleaned with an alcohol swab, letting the area air dry for about 10 seconds. A lancet is used to puncture the patient's skin in the cleaned area, and then blood drops are collected in the blood collection tube (i.e., K₂EDTA collection tube, optionally with a preservative agent), using the lip of the tube to collect the blood drops as they form. The collection tube is rocked or tapped gently or pushed downward to allow the blood drops to settle in the bottom of the tube with the anti-clotting agent. Once the desired amount of blood sample is collected (e.g., approximately 500 μL), the collection tube is sealed and rotated end over end several times to ensure the blood is properly mixed with the anti-clotting agent and preservative agent. The collection tube and re-capped lancet are placed in two slots in a foam insert within an aluminum container. A foam cap is placed on top of the foam insert containing the lancet and collection tube, and then the screw cap to the aluminum canister is replaced. The aluminum canister is placed in an outer canister with a screw top. The canister is placed into an overnight envelope or U.S. Postal Service Express Mail envelope, and the package is mailed to the testing facility for analysis.

Blood samples from adult patients are collected in a 3 ml EDTA collection tube, K₂EDTA collection tube, or lithium-heparin collection tube. Pediatric patient blood samples may be collected in EDTA microtubes—Minimum of 200 μL (0.2 mL). Suitable samples for the described methods have been stored at ambient temperature for twenty-four hours or less, at 4° C. for seven days or less, or at −20° C. for up to six months. Samples that are clotted, have inadequate volume, plasma, serum, or that have been at ambient temperature for greater than twenty-four hours are not suitable for the methods described herein. Temperature stability studies are performed to determine stability of each analyte at various environmental temperatures for various lengths of time to establish criteria for rejection of samples.

### Example 2

### Therapeutic Drug Monitoring of Immunosuppressants (TDM-IDS) in a Small Volume Blood Sample

### Sirolimus Levels

Sirolimus levels are measured using LC-MS/MS (Tandem Mass Spectrometry) or high performance liquid chromatography coupled to electro-spray ionization tandem mass spectrometry (HPLC-ESI-MS/MS) under standard conditions known in the art, and results of this testing are available to the patient or healthcare provider usually within twenty-four hours of receipt of the sample.

The testing facility provides reference lab service for the immunosuppressant drug monitoring of Rapamycin (Sirolimus, RAPAMUNE®—Wyeth) for transplant programs. The facility utilizes high performance liquid chromatography coupled to electro-spray ionization tandem mass spectrometry (HPLC-ESI-MS/MS or LC-MS/MS) as the definitive quantitative method to accurately determine blood levels of rapamycin. Tandem mass spectrometry has been demonstrated in numerous studies to be the method of choice for immunosuppressant drug monitoring of rapamycin. In addition, the multiplex capability of LC-MS/MS allows for simultaneous measurement of other prescribed immunosuppressants, such as Tacrolimus (PROGRAF®—Astellas), Cyclosporin A (SANDIMMUNE® & NEORAL®—Novartis), Mycophenolic Acid (CELLCEPT®—Roche, MYFORTIC®—Novartis), or Everolimus (CERTICAN®—Novartis), if desired, as well as allowing for the simultaneous monitoring of renal and/or hepatic function by splitting the sample and analyzing using standard methods (See, e.g., Alfawassermann ACE Clinical Chemistry System Operator's Manual, August 2005 revision). By use of overnight courier services and dedicated assay procedures in the testing facility, the turn-around time is suitable for acute therapeutic management, as well as routine monitoring of transplant patients.

### Tacrolimus Levels

Tacrolimus levels are measured using LC-MS/MS (Tandem Mass Spectrometry) or high performance liquid chromatography coupled to electro-spray ionization tandem mass spectrometry (HPLC-ESI-MS/MS) under standard conditions known in the art, and results of this testing are available to the patient or healthcare provider usually within twenty-four hours of receipt of the sample.

The testing facility provides reference lab service for the immunosuppressant drug monitoring of Tacrolimus (PROGRAF®—Astellas) for transplant programs. The facility utilizes high performance liquid chromatography coupled to electro-spray ionization tandem mass spectrometry (HPLC-ESI-MS/MS or LC-MS/MS) as the definitive quantitative method to accurately determine blood levels of tacrolimus. Tandem mass spectrometry has been demonstrated in numerous studies to be the method of choice for immunosuppressant drug monitoring of tacrolimus. Tandem mass spectrometry does not react with drug metabolites and is not affected by liver function or sample preparation as is the standard immunoassay test used. The standard immunoassay is nonspecific and has been shown to give erroneously high drug levels in a variety of situations which may lead to under dosing and risk of organ rejection.

In addition, the multiplex capability of LC-MS/MS allows for simultaneous measurement of other prescribed immunosuppressants, such as Sirolimus, Cyclosporin A, Mycophenolic Acid, or Everolimus, if desired, as well as allowing for the simultaneous monitoring of renal and/or hepatic function by splitting the sample and analyzing using standard methods (See, e.g., Alfawassermann ACE Clinical Chemistry System Operator's Manual, August 2005 revision). By use of overnight courier services and dedicated assay procedures in the testing facility, the turn-around time is suitable for acute therapeutic management, as well as routine monitoring of transplant patients.

### Cyclosporin A

Cyclosporin A levels are measured using LC-MS/MS (Tandem Mass Spectrometry) or high performance liquid chromatography coupled to electro-spray ionization tandem mass spectrometry (HPLC-ESI-MS/MS) under standard conditions known in the art, and results of this testing are available to the patient or healthcare provider usually within twenty-four hours of receipt of the sample.

The testing facility provides reference lab service for the immunosuppressant drug monitoring of Cyclosporin A for transplant programs. The facility utilizes high performance liquid chromatography coupled to electro-spray ionization tandem mass spectrometry (HPLC-ESI-MS/MS or LC-MS/MS) as the definitive quantitative method to accurately determine blood levels of cyclosporin A. Tandem mass spectrometry has been demonstrated in numerous studies to be the method of choice for immunosuppressant drug monitoring of cyclosporine A. Tandem mass spectrometry does not react with drug metabolites and is not affected by liver function or sample preparation as is the standard immunoassay test used. Further, the standard immunoassay test is nonspecific and has been shown to give erroneously high drug levels in a variety of situations which may lead to under dosing and risk of organ rejection.

In addition, the multiplex capability of LC-MS/MS allows for simultaneous measurement of other prescribed immunosuppressants, such as Sirolimus, Tacrolimus, Mycophenolic Acid, or Everolimus, if desired, as well as allowing for the simultaneous monitoring of renal and/or hepatic function by splitting the sample and analyzing using standard methods (See, e.g., Alfawassermann ACE Clinical Chemistry System Operator's Manual, August 2005 revision). By use of overnight courier services and dedicated assay procedures in the testing facility, the turn-around time is suitable for acute therapeutic management, as well as routine monitoring of transplant patients.

### Everolimus and MPA

Levels of the immunosuppressant drugs everolimus and mycophenolic acid (MPA) have not yet been routinely monitored in transplant patients. Everolimus is currently awaiting final FDA approval in the United States, and MPA assays are under development for routine testing of transplant patients. However, the methods described herein are useful in the immunosuppressant drug monitoring of everolimus and MPA.

The everolimus and MPA levels are measured using LC-MS/MS (Tandem Mass Spectrometry) or high performance liquid chromatography coupled to electro-spray ionization tandem mass spectrometry (HPLC-ESI-MS/MS) under standard conditions known in the art, and results of this testing are available to the patient or healthcare provider usually within twenty-four hours of receipt of the sample.

A testing facility provides reference lab service for the immunosuppressant drug monitoring of everolimus and MPA for transplant programs. The facility utilizes high performance liquid chromatography coupled to electro-spray ionization tandem mass spectrometry (HPLC-ESI-MS/MS or LC-MS/MS) as the definitive quantitative method to accurately determine blood levels of these drugs. In addition, the multiplex capability of LC-MS/MS allows for simultaneous measurement of other prescribed immunosuppressants, such as Sirolimus, Tacrolimus, Cyclosporin A, and Everolimus, if desired, as well as allowing for the simultaneous monitoring of renal and/or hepatic function by splitting the sample and analyzing using standard methods (See, e.g., Alfawassermann ACE Clinical Chemistry System Operator's Manual, August 2005 revision). By use of overnight courier services and dedicated assay procedures in the testing facility, the turn-around time is suitable for acute therapeutic management, as well as routine monitoring of transplant patients.

### Example 3

Analysis of Renal and/or Hepatic Function Using a Small Volume Blood Sample

The renal function and/or hepatic function of a patient is determined using a small volume blood sample. Blood samples from adult patients are collected in a 3 mL EDTA collection tube, K₂EDTA collection tube, or lithium-heparin collection tube as described above. Pediatric patient blood samples may be collected in 0.2 mL EDTA microtubes. Suitable samples for the described methods have been stored at ambient temperature for about twenty-four hours or less, at about 4° C. for seven days or less, or at −20° C. for up to three months. Temperature stability studies are performed to determine stability of each analyte at various environmental temperatures for various lengths of time to establish criteria for rejection of samples. Generally, samples that are clotted, have inadequate volume, plasma, serum, or that have been at ambient temperature for greater than about twenty-four hours in the absence of a preservative agent are not suitable for the methods described herein.

The renal function is monitored by determining the level of creatinine, blood urea nitrogen (BUN), or both in the sample using standard methods (See, e.g., Alfawassermann ACE Clinical Chemistry System Operator's Manual, August 2005 revision). If the kidney function is normal, the blood creatinine levels fall within about 0.6 and about 1.2 mg/dL or about 53 to about 106 μmol/L for men, within about 0.5 and about 1.1 mg/dL or about 44 to about 97 μmol/L for women, within about 0.5 and about 1.0 mg/dL for teens, within about 0.3 and about 0.7 mg/dL for children, and within about 0.3 and about 1.2 mg/dL for newborns. If the kidney function is normal, the BUN to creatinine ratio is between about 10:1 and about 20:1 for patients over 12 months of age and up to about 30:1 for patients less than 12 months of age.

The patient's hepatic function is monitored by determining the levels of AST, ALT, AP, TBIL, direct bilirubin, or a combination thereof using standard methods (See, e.g., Alfawassermann ACE Clinical Chemistry System Operator's Manual, August 2005 revision). If liver function is normal, the AST level is within about 10 and about 40 IU/L; the ALT level is within about 5 and about 40 IU/L; the AP level is within about 30 and about 120 IU/L; the TBIL is within about 2 to about 14 μmol/L; and the direct bilirubin level is less than or about 4 μmol/L.

### Example 4

Therapeutic Drug Monitoring of Immunosuppressant Levels and Monitoring of Hepatic and Renal Function from a Single Small Volume Blood Sample

An adult male patient received rapamycin, tacrolimus, and cyclosporine A as part of his treatment regimen. The patient collected an approximately 500 μL blood sample from a fingerstick. The small volume blood sample was collected as described above, using a K₂-EDTA collection tube, and timely transported to the testing facility. At the testing facility, the whole blood sample in K₂-EDTA was split into 3 fractions: (1) 50 μL for immunosuppressant drug monitoring using tandem mass spectrometry, (2) 50 μL for complete blood count by flow cytometry, and (3) the remainder for various other tests.

The first fraction was prepared by adding 200 μL of ZnSO₄ (zinc sulfate) to the fraction, and the cells were lysed by vortexing for 10 seconds. Then 500 μL of a solution containing acetonitrile and internal reference standards (ascomycin: 2 nanograms (ng)/ml, along with cyclosporin D 25 ng/ml) was added, and the sample was mixed by vortexing for 20 seconds. The tubes were then spun down for 4 minutes at 14,000×g (14,000 times gravity). The supernatant was decanted into vials for assay by mass spectrometry. The sample was analyzed for levels of rapamycin, tacrolimus, and cyclosporin A using tandem mass spectrometry, and the results are shown in Table 1 below. The results for these assays are within the range of sensitivity discussed above.

The second fraction, used for complete blood count, does not require any further preparation before the flow cytometry assay. A first 25 μL was used to determine the blood counts for the blood sample, reserving a second 25 μL for repeat testing if necessary. This assay determined the white blood cell count (WBC), red blood cell count (RBC), hemoglobin (Hgb), hematocrit (Hct), mean cell volume (MCV), mean cell hemoglobin (MCH), mean cell hemoglobin concentration (MCHC), platelet (PLT), lymphocyte percent (LYM %), mixed cell type percent (MXD %), neutrophil percent (NEUT %), lymphocyte number (LYM#), mixed cell type percent (MXD#), and neutrophil number (NEUT#). The results of this assay are shown below in Table 1.

The third fraction was centrifuged for 3 minutes at 14,000×g, and the supernatant was decanted into reaction cells for the ACE Alera chemistry analyzer. This equipment uses standard photometric assay methods. The renal function of this patient's blood sample was analyzed by determining the level of creatinine and blood urea nitrogen in the sample, and the results are shown in Table 1. The results for these assays are within the range of sensitivity discussed above. The hepatic function of this patient's blood sample was analyzed by determining the level of aspartate transaminase (AST), alanine transaminase (ALT), and alkaline phosphatase (ALP), and the results are shown in Table 1. The results for these assays are within the range of sensitivity discussed above.

TABLE 1

| Assay | Result | Units |
|---|---|---|
| Rapamycin | 5.10 | μg/L |
| Tacrolimus | 6.80 | μg/L |
| Cyclosporine A | 230.00 | μg/L |
| Creatinine | 3.10 | mg/dL |
| BUN | 29.00 | mg/dL |
| AST | 68.00 | U/L |
| ALT | 79.00 | U/L |
| ALP | 38.00 | U/L |
| WBC | 2.70 | ×10E+3/μL |
| RBC | 2.66 | ×10E+6/μL |
| Hgb | 7.20 | g/dL |
| Hct | 42.00 | % |
| MCV | 79.70 | fL |
| MCH | 27.10 | pg |
| MCHC | 34.00 | g/dL |
| PLT | 56.00 | ×10E+3/μL |
| LYM % | 24.40 | % |
| MXD % | 6.00 | % |
| NEUT % | 69.60 | % |
| LYM# | 0.70 | ×10E+3/μL |
| MXD# | 0.20 | ×10E+3/μL |
| NEUT# | 1.80 | ×10E+3/μL |

An adult female patient received rapamycin, tacrolimus, and cyclosporine A as part of her treatment regimen. The patient collected an approximately 500 μL blood sample from a fingerstick. The small volume blood sample was collected using a K₂-EDTA collection tube, transported to the testing facility, and divided into three fractions as described above. Each fraction was prepared as described above for the adult male patient, and the same immunosuppressant drug monitoring of immunosuppressants, complete blood count, assays for renal function, and assays for hepatic function were performed. The results are shown in Table 2.

TABLE 2

| Assay | Result | Units |
|---|---|---|
| Rapamycin | 3.10 | μg/L |
| Tacrolimus | 10.80 | μg/L |
| Cyclosporine A | 123.00 | μg/L |
| Creatinine | 2.40 | mg/dL |
| BUN | 22.00 | mg/dL |
| AST | 44.00 | U/L |
| ALT | 27.00 | U/L |
| ALP | 91.00 | U/L |
| WBC | 2.90 | ×10E+3/μL |
| RBC | 2.66 | ×10E+6/μL |
| Hgb | 7.20 | g/dL |
| Hct | 36.00 | % |
| MCV | 81.00 | fL |
| MCH | 22.10 | pg |
| MCHC | 36.00 | g/dL |
| PLT | 72.00 | ×10E+3/μL |
| LYM % | 20.60 | % |
| MXD % | 6.00 | % |
| NEUT % | 73.40 | % |
| LYM# | 0.90 | ×10E+3/μL |
| MXD# | 0.20 | ×10E+3/μL |
| NEUT# | 1.60 | ×10E+3/μL |

### Example 5

Therapeutic Drug Monitoring of Immunosuppressant Levels and Monitoring of Hepatic and Renal Function from a Blood Spot

An adult patient collects an approximately 500 μL blood sample from a fingerstick. The small volume blood sample is collected by collecting several different blood spots on suitable filter paper and allowing the spots to dry. The sample is transported to the testing facility where it is initially processed.

A predetermined area of dried blood on the filter paper or other media is selected, corresponding to a known blood volume, as published by CDC references (e.g., ⅛ inch spot=8 microliters of blood). The blood is extracted using solvents known in the art for the purposes of analyzing the desired analytes, including acetonitrile solvent with or without internal standards and/or methanol extraction solvent. The extract is then centrifuged to remove particulates and subjected to analysis by HPLC-MS/MS, clinical chemistry analyzer systems, and other instrument systems amenable to use of dried blood extract. The sample is analyzed for the level of one or more immunosuppressant drugs and for the renal and/or hepatic function of the patient as described herein to determine the sufficiency of the current treatment regimen. The CBC hematology analysis is unlikely to work with this type of blood sample. The results are transmitted to the patient and/or to the healthcare provider.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. A method for monitoring a immunosuppressant drug level and renal function, hepatic function, or a combination thereof in a patient, comprising:
obtaining a small volume blood sample from a patient;
determining the level of at least one immunosuppressant drug in the small volume blood sample; and
determining the level of a second immunosuppressant drug or analyzing the renal function, the hepatic function, or a combination thereof in the small volume sample.

2. The method of claim 1, wherein the level of the at least one immunosuppressant drug is determined using a liquid chromatography tandem mass spectrometry (LC-MS/MS) procedure.

3. The method of claim 1, wherein the at least one immunosuppressant drug is selected from the group consisting of cyclosporin, tacrolimus, sirolimus, mycophenolic acid, and everolimus.

4. The method of claim 1, wherein the levels of the at least one immunosuppressant drug and the second immunosuppressant drug are determined simultaneously.

5. The method of claim 4, wherein the renal function, hepatic function, or a combination thereof is analyzed from the small volume blood sample.

6. The method of claim 1, wherein the small volume blood sample is a fingerprick sample or an earlobe sample.

7. The method of claim 6, wherein the patient obtains his or her own small volume blood sample.

8. The method of claim 1, wherein the renal function is analyzed by determining the glomerular filtration rate, the creatinine clearance rate, the level of creatinine, the level of urea, or a combination thereof.

9. The method of claim 8, wherein the renal function is analyzed by determining the level of creatinine, urea, or both in the sample.

10. The method of claim 1, wherein the hepatic function is analyzed by determining the levels of aspartate transaminase (AS T), alanine transaminase (ALT), alkaline phosphatase (AP), total bilirubin (TBIL), direct bilirubin, gamma glutamyl transpeptidase (GGT), 5′ nucleotidase (5′NTD), serum glucose, lactate dehydrogenase, or a combination thereof.

11. The method of claim 10, wherein the hepatic function is analyzed by determining the levels of AST, ALT, AP, TBIL, direct bilirubin, or a combination thereof.

12. The method of claim 1, wherein the patient has had or is going to have an organ transplant.

13. The method of claim 12, wherein the organ is selected from the group consisting of liver, kidney, heart, lung, and bone marrow.

14. A kit for use in methods for monitoring a immunosuppressant drug level and renal function, hepatic function, or a combination thereof in a patient, comprising:
a fingerprick device for obtaining a small volume blood sample;
a container for collecting the small volume blood sample comprising an anti-clotting agent and optionally a preservative agent;
a package for the transport of the small volume blood sample; and
instructions for collecting and packaging the small volume blood sample, and for transporting the small volume blood sample.

15. The kit of claim 14, wherein the fingerprick device is a lancet.

16. The kit of claim 14, wherein the anti-clotting agent is K₂-EDTA.

17. A system for monitoring a immunosuppressant drug level and renal function, hepatic function, or a combination thereof in a patient, comprising:
obtaining a small volume blood sample from the patient;
placing the small volume blood sample in a container for transport to a testing facility that analyzes the small volume blood sample for the level of at least one immunosuppressant drug and renal function, hepatic function, or a combination thereof; and
receiving results from the testing facility for the level of at least one immunosuppressant drug and renal function, hepatic function, or a combination thereof.

18. The system of claim 17, wherein the patient obtains his or her own blood sample.

19. The system of claim 17, wherein the small volume blood sample is a fingerprick sample or an earlobe sample.

20. The system of claim 17, wherein the patient receives the results directly from the testing facility.

21. The system of claim 17, wherein the patient receives the results from a health care provider that received the results from the testing facility.

22. The system of claim 17, wherein the small volume blood sample is transported to the testing facility by a mail service.
